(19) United States
(12) Patent Application Publication
(10) Pub. No.: US 2023/0173133 A1
(43) Pub. Date: Jun. 8, 2023

(54) ATMOSPHERIC PLASMA FILTER

(71) Applicant: Atmospheric Plasma Solutions, Inc., Cary, NC (US)

(72) Inventor: Peter J. Yancey, Cary, NC (US)

(21) Appl. No.: 17/911,708

(22) PCT Filed: Mar. 17, 2021
(86) PCT No.: PCT/US21/22859

§ 371 Date: Sep. 15, 2022

Related U.S. Application Data
(60) Provisional application No. 62/991,507, filed on Mar. 18, 2020.

(51) Int. Cl. A61L 9/22; H05H 1/24; B01D 53/32; B01D 53/88; B01D 53/00; B01D 39/20; B01D 53/86; A61L 9/20; B01J 21/06; B01J 21/08; B01J 35/06; B01J 35/00

(52) U.S. Cl. CPC A61L 9/22; H05H 1/2431; B01D 53/326; B01D 53/885; B01D 53/007; B01D 39/2017; B01D 53/8696; A61L 9/205; B01J 21/063; B01J 21/08; B01J 35/06; B01J 35/004; H05H 2245/15; H05H 1/0006

(57) ABSTRACT

A plasma filter for treating a gas flow therethrough. The filter has a dielectric barrier plasma electrode assembly including a plurality of electrodes having a dielectric barrier layer coated thereon. The dielectric barrier plasma electrode assembly is configured to produce an atmospheric pressure plasma, A filtration medium is disposed on or between the electrodes, and a photocatalytic material is formed on surfaces of the filtration medium. Upon operation of the plasma filter, the plasma infiltrates voids in the filtration medium, and the gas flow through the filtration medium a) is exposed to reactive species of the plasma, b) interacts with the catalytic material, and c) is exposed to light generated from the plasma.

### CROSS REFERENCE TO RELATED APPLICATIONS

This application is related to and claims priority to U.S. Ser. No. 62/991,507 filed Mar. 18, 2020, entitled “Atmospheric Plasma Filter,” the entire contents of which are incorporated herein by reference.

### BACKGROUND OF THE INVENTION

### Field of Invention

The invention relates to the use of a dielectric barrier plasma at atmospheric pressure for purification of a gas stream.

### Discussion of the Background

Plasmas have been used extensively in a wide variety of industrial and high technology applications including, for example, semiconductor fabrication, various surface modifications, and coatings of reflective films for window panels and compact disks. Plasmas ranging in pressure from high vacuum (<0.1 mTorr) to several Torr to atmospheric pressures are common. It is also known to use plasma, typically O₂ plasmas, as a way of removing hydrocarbon and other organic surface contaminants from various substrates.

Plasmas have the ability to create large amounts of reactive chemical species, such as ions, radicals, electrons, excited-state (e.g., metastable) species, molecular fragments, photons, and the like. Plasmas also have the ability to create fluxes of radiation (e.g., ultraviolet, xays, gamma rays electrons and other excited-state species. Plasmas can generate photons including ultraviolet photons that have enough energy to initiate photochemical and photocatalytic reaction paths in biological and other materials that are irradiated by the plasma.

Plasmas have been used for decontamination and sterilization. In general, both disinfection and sterilization to remove pathogens. The key to distinguishing the two techniques is the endospore. Removing pathogens but leaving endospores is considered disinfection, while completely destroying both endospores and pathogens is considered sterilization. Biological decontamination, disinfection, and/or sterilization have a broad array of applications including medical equipment and device sterilization, food production and preservation, and preparation of consumer goods. Chemicals, heat, high-energy electron beams, and X-ray or gamma-ray irradiation systems are presently used for sterilization. Each of these systems has trade-offs due to the cost, efficiency, immobility, electric power requirements, toxic waste, personal hazard and the time required for sterilization or decontamination.

Additionally, atmospheric plasmas have been used for antimicrobial sterilization and chemical breakdown (digestion). Prior work has reported on the effective use of the glow discharge at atmospheric pressure to inactivate bacterial cells. This work illustrated the destruction of Pseudomonas fluorecens by atmospheric pressure plasma using suspensions of the bacteria in Petri dishes placed on a dielectric covered lower electrode. The electrodes were placed within a chamber containing mostly helium with an admixture of air. This work obtained full destruction of concentrations of 4×10⁶/ml in less than 10 min. Using a similar discharge, prior work also reported the inactivation of Bacillus subtilis spores in air. In addition, Aspergillus niger spores, Bacillus subtilis, and a variety of other gram-negative as well as gram-positive bacteria were inactivated successfully by many researchers using the DBD-based diffuse-glow discharge.

It is also known that some toxic organic compounds when in contact with properly electrically excited semiconductor surfaces can undergo oxidation, thus degrading the toxins into non-toxic substances. When certain semiconductors absorb UV light of energy greater than their bandgap (3.2-3.3 eV for TiO₂, λ less than or equal to 380 nm), electron-hole pairs are generated; if the created holes can reach the surface prior to recombination, they are available for redox reactions with species adsorbed on the semiconductor surface. In some cases the degradation is believed to occur either by direct oxidation of the pollutants or by the formation of reactive radicals on the surface such as OH. or other reactive species that may be formed on such a surface with the appropriate surface layers available for activation. The details of the mechanisms responsible for such photo-degradation are not fully understood, but many experiments demonstrate the feasibility of such photocatalytic systems.

A semiconductor of choice for photocatalytic degradation has been the anatase phase of titanium oxide. While the photocatalytic abilities of anatase are well known, it is desirable to increase its photoreactivity for improved degradation of contaminants. One approach used was the addition of dopants to decrease the bandgap, and thus increase the cut-off wavelength for electron-hole pair generation. Co-catalysts can also be used to increase photoreactivity. The role of the co-catalyst is to facilitate the particular reaction (oxidation or reduction, depending on specific conditions) that is rate limiting. Increasing the rate of this reaction results in an overall increase in reactivity as demonstrated with the addition of Pt and Ir co-catalysts to TiO₂ anatase. Furthermore, by adding a small positive bias to an anatase coated conductive substrate, photocatalytic enhancement by as much as 90% has been achieved. The substrate, behaving as the anode, forcibly removes electrons (from photo-generated electron-hole pairs) from the anatase, thus suppressing undesired electron-hole recombination. The doping of anatase combined with the use of a co-catalyst and an applied bias can result in a photocatalyst that is significantly more effective in the degradation of contaminants.

In general, the following references (the entire contents of which are incorporated by reference) detail the state of the art in plasma treatments of contaminated gas streams and the use of catalytic materials for this purpose:
1. U.S. Pat. No. 9,117,636, entitled “Plasma Catalyst Chemical Reaction Apparatus.”
2. U.S. Pat. No. 10,194,672, entitled “Reactive Gas, Reactive Gas Generation System and Product Treatment Using Reactive Gas.”
3. US Pat. Appl. Publ. No. 2011/0180149, entitled “Single Dielectric Barrier Discharge Plasma Actuators with In-Plasma Catalysts and Method of Fabricating the Same.”
4. US Pat. Appl. Publ. No. 2013/0330229, entitled “Plasma System for Air Sterilization.”
5. M. Laroussi, “Sterilization of Tools and Infectious Waste by Plasmas,” Bulletin of American Physical Society Division of Plasma Physics 40, No. 11, 1685-1686 (1995).
6. M. Laroussi, “Sterilization of Contaminated Matter with an Atmospheric Pressure Plasma.” IEEE Transactions on Plasma Science 24, 1188-1191 (1996).
7. Kelly-Wintenberg, T. C. Montie, C. Brickman, J. R. Roth, A. K. Carr, K. Sorge, L. C. Wadworth, and P. P. Y. Tsai, “Room Temperature Sterilization of Surfaces and Fabrics with a One Atmosphere Uniform Glow Discharge Plasma.” IEEE Transactions on Plasma Science 20, 69-74 (1998).
8. J. G. Birmingham and D. J. Hammerstrom, “Bacterial Decontamination Using Ambient Pressure Nonthermal Discharges.” IEEE Transactions on Plasma Science 28, 51-55 (2000).
9. F. Trompeter. W. J. Neff, O. Franken, M. Heise, M. Neiger, S. Liu, G. J. Pietsch, A. B. Saveljew, “Reduction of Bacillus subtilis and Aspergillus niger Spores Using Nonthermal Atmospheric Gas Discharges,” IEEE Transactions on Plasma Science 30, 1416-1423 (2002).
10. N. S Panikov, S. Paduraru, R. Crowe, P. J. Ricatto, C. Christodoulatos, and K. Becker, “Destruction of Bacillus subtilis Cells Using an Atmospheric-Pressure Capillary Plasma Electrode Discharge.” IEEE Transactions on Plasma Science 30, 1424-1428 (2002).
11. R. L. Pozzo, M. A. Baltanas, and A. E. Cassano, “Supported Titanium Oxide as Phototcatahvst in Water Decontamination: State of the Art,” Catalysis Today 39, 219-231 (1997).
12. M. Anpo, “Utilization of TiO2 Photocatalysts in Green Chemistry,” Pure Applied Chemistry, 72 (7) 1265-1270 (2000).
13. C. A. Linkous, G. J. Carter, D. B. Locuson, A. J. Ouellette, D. K. Slattery, and L. A. Smitha. “Photocatalytic Inhibition of Algae Growth Using TiO2, WO3, and Cocatalyst Modifications,” Environmental Science & Technology 34 (22), 4754 (2000).
14. Kim, Kwang-Wook, Eil-Hee Lee, Young-Jun Kim, Mi-Hye Lee and Dong-Woo Shin. “A Study on Characteristics of an Electrolytic-Photocataytic Reactor Using an Anode Coated with TiO2,” Journal of Photochemistry and Photobiology A: Chemistry. ARTICLE IN PRESS.
15. P. Maness, S. Smolinksi, D. M. Blake, Z. Huang, E. J. Wolfrum, and W. A. Jacobv, “Bactericidal Activity of Photocatalytic TiO2 Reaction: Toward an Understanding of Its Killing Mechanism,” Applied and Environmental Microbiology 65 (9), 4094-4098 (1999).
16. F. Massines and G. Gouda. “A comparison of polypropylene-surface treatment by filamentary, homogeneous and glow discharge in helium at atmospheric pressure”. Journal of Applied Physics: D 31, 3411-3420 (1998).
17. P. Setlow, “Spores of Bacillus Species: Mechanisms of their Resistance to and Killing by Decontamination/Sterilization Agents.” 24th Army Science Conference Papers (2004).

### SUMMARY

In one embodiment of the invention, there is provided a plasma filter for treating a gas flow and any contaminants entrained in the gas flow therethrough. The plasma filter has a dielectric barrier plasma electrode assembly including a plurality of electrodes having a dielectric barrier layer coated thereon. The dielectric barrier plasma electrode assembly is configured to produce an atmospheric pressure plasma. A filtration medium is disposed on or between the electrodes of the supporting grid, and a catalytic material is formed on surfaces of the filtration medium. Upon operation of the plasma filter, the plasma infiltrates voids in the filtration medium, and the gas flow through the filtration medium a) is exposed to reactive species of the plasma, b) interacts with the catalytic material, and c) is exposed to light (e.g., ultraviolet light) generated from the plasma.

In one embodiment of the invention, there is provided a method for sterilization (or otherwise disinfection) of a gas flow using the plasma filter described above.

In one embodiment of the invention, there is provided a plasma filter and downstream converter system for treating a gas flow therethrough, comprising the plasma filter described above, a coupling unit connected to a plasma filter enclosure of the plasma filter and a converter connected to the coupling unit and containing for example a catalyst for converting for example ozone into oxygen (a more benign species). Upon operation of the plasma filter and the converter, the gas flow and any entrained contaminants passing through the filtration medium a) is exposed to reactive species of the plasma, b) interacts with the catalytic material on the filtration medium, and c) is exposed to ultraviolet light generated from the plasma. Effluents from the plasma filter are supplied to the converter to convert species in the effluents into more benign species.

It is to be understood that both the foregoing general description of the invention and the following detailed description are exemplary, but are not restrictive of the invention.

### BRIEF DESCRIPTION OF THE FIGURES

A more complete appreciation of the invention and many of the attendant advantages thereof will be readily obtained as the same becomes better understood by reference to the following detailed description when considered in connection with the accompanying drawings, wherein:

FIG. 1 is a schematic of an atmospheric plasma filter according to one embodiment of the invention;

FIG. 2 is a schematic of an emission spectrum from a plasma generated by the atmospheric plasma filter of the invention;

FIG. 3 is a schematic of another emission spectrum from a plasma generated by the atmospheric plasma filter of the invention;

FIG. 4 is a schematic of x-ray diffraction data taken from different sol gel deposited TiO₂ layers;

FIG. 5 is a schematic of a dielectric barrier plasma electrode assembly according to an embodiment of the invention;

FIG. 6 is a schematic of another emission spectrum from a plasma generated by the atmospheric plasma filter of the invention;

FIG. 7A is a schematic of an atmospheric plasma filter/ozone converter according to one embodiment of the invention;

FIG. 7B is a schematic of air handler of the present invention including an atmospheric plasma filter and ozone converter in an annular configuration;

FIG. 7C is a cross-section of the assembly shown in FIG. 7B taken along axis C; and

FIG. 8 is a flow chart detailing a method of the present invention for treatment of a gas flow.

### DETAILED DESCRIPTION OF THE INVENTION

There is an increased urgency in the development of protective equipment capable of preventing or neutralizing the potential devastation brought on by a terrorist attack consisting of biological and/or chemical weaponry, and especially in today's response to the present coronavirus. Italy reports that a high percentage of the first line staff who have attended to so many sickened with the coronavirus are themselves now infected, presenting a new dilemma for the health care community of having enough coronavirus-free staff to continue to treat the growing numbers of those infected. The equipment fashioned must be capable of defending military personnel, emergency responders, hospital staff, and the public from these escalating threats. Thus, there exists a critical need for improved air filtration technologies that supersede the protective capabilities of currently available air filtration systems.

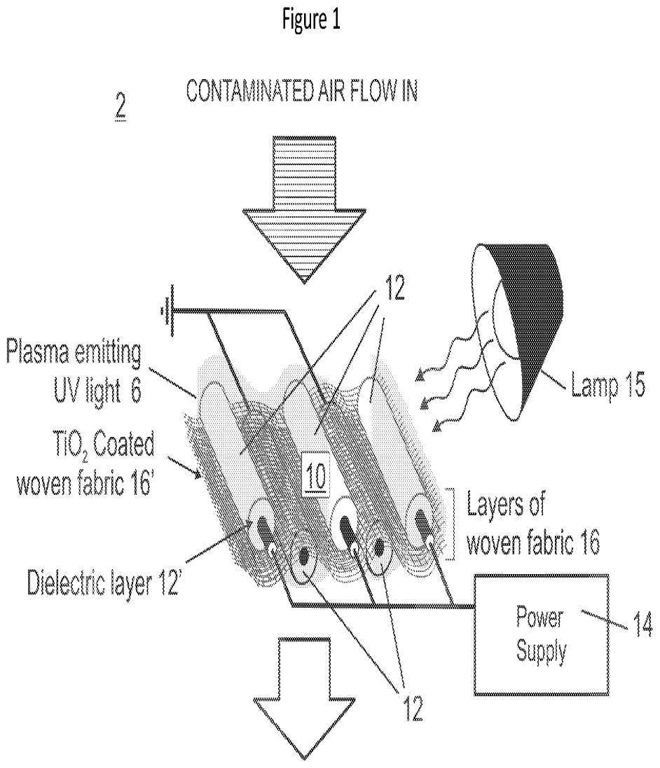

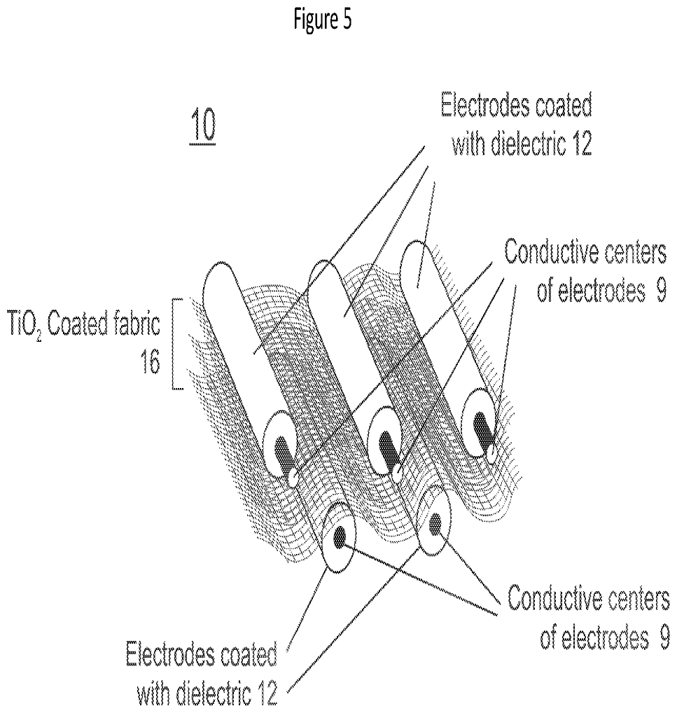

FIG. 1 is a schematic of a plasma filter according to one embodiment of the invention for sterilization (and/or disinfection) of a gas flow therethrough containing harmful agents. As shown in FIG. 1, the plasma filter 2 has a dielectric barrier plasma electrode assembly 10 including a plurality of longitudinally extending electrodes 12 having a dielectric barrier layer 12′ coated thereon. (FIG. 5 to be discussed in more detail later shows the conductive centers 9 of electrodes 12.) In the exemplary configuration shown in FIG. 1, there is a top layer of electrodes 12 connected to a power supply 14, and there is a bottom layer of electrodes 12 connected to ground. This configuration produces a plasma in the region between the top layer and the bottom layer of electrodes, although the plasma diffuses from this region. Other power configurations can be used than the one shown in FIG. 1. For example, alternating electrodes in the layers of electrodes could be powered with the other electrodes being grounded. Alternatively, individual electrodes of the layers of electrodes may be driven separately and not necessarily with aground reference, or individual electrodes of the layers of electrodes may be driven separately, for example out of phase with one another.

In one embodiment, the plurality of longitudinally extending electrodes 12 forms a supporting grid. A filtration mat 16 (for example layers of a woven fabric) is disposed on or between the electrodes 12 forming the supporting grid holding the filtration mat (or medium) 16, and a catalytic material 16′ (e.g., TiO₂ anatase) is formed on strand surfaces of the filtration mat 16. In one embodiment, although not essential to operation, the plasma 6 infiltrates voids in the filtration mat, and the gas flow (i.e., the contaminated air flow) passes through the filtration mat 16. The passage of the air flow through the plasma filter 2 and especially through the filtration mat 16 means that the contaminated air:

a) is exposed to reactive species of the plasma discharge,

b) interacts with the catalytic material (on exterior and interior surfaces of the filtration medium), and

c) is exposed to ultraviolet light generated from the plasma.

Thus, the FIG. 1 configuration in one embodiment of the invention provides for a multifunctional approach to air filtration technology, where a combination of the following is used; ultraviolet light (UV) radiation, atmospheric plasma decontamination, photocatalysis, and physical filtration. In other words, this approach combines an atmospheric plasma, a physical fiberglass filter media, a photocatalyst on the surfaces (exterior and interior) of the fiberglass filter media, where each component contributes to the overall functionality of the plasma filter 2. As used herein, atmospheric plasma refers to plasma operating at, slightly below, or slightly above atmospheric pressure and refers typically to operation at pressures between 500 and 1500 Torr absolute.

For example, providing the atmospheric plasma in air produces chemically reactive species, energetically charged particles, and germicidal ultraviolet light having %<260 nm, where the light (for example a majority of the light 50%, 60%, 70% or greater) is UV light below a wavelength of 380 nm which can photocatalytically activate TiO₂ anatase or other suitable photocatalyst. Activation of the TiO₂ anatase by the plasma generated light means that OH. radicals are produced for oxidation of any bacterial or viral agents in the gas flow or in general oxidation of chemical and biological agents in the gas flow. Optionally, the plasma filter 2 with its UV light and its reactive plasma species and its photocatalytically activated surfaces of TiO₂ can be used to treat various gaseous species such as nitric oxides (NOₓ) (a class of known air pollutants) and convert them into more benign species or other species, such as HNO₃, that may be more easily abated using conventional catalytic or other mechanisms. Furthermore, conventional catalyst such as platinum, palladium, rhodium, manganese or other catalysts can be included along with TiO₂ anatase in the fiber mat 12 to assist in the abatement of undesirable gaseous species in the airstream. Finally, the fiber mat 12 represents a physical barrier for filtering the gas flow which, in combination with the above, both stops and neutralizes the chemical and biological agents in the gas flow.

While TiO₂ anatase is used here to illustrate the invention, other photocatalytic materials included doped TiO₂ and co-catalysts can be used, such as, the addition of Pt and Ir co-catalysts to TiO₂ anatase.

Thus, in one embodiment of the invention, the plasma filter 2 combines the sterilizing mechanisms of atmospheric plasma (chemically reactive species and energetic charged particles) with the UV light driven photocatalytic activation of TiO₂ anatase (or other photocatalytic material) on a filter media in a non-stagnant air filtration device. Prior to this invention, anatase had been typically activated “upon excitation by light whose wavelength is below 380 nm, with the photon energy generating the desired electron hole pairs on the TiO₂ anatase surface.” In those prior art systems, the UV light “activation sources” were limited by absorption, scattering, and proximity to the activated media, which the present invention addresses especially with the plasma infiltrating voids in the filtration mat 12 where it activates the TiO₂ anatase surfaces.

Viewed differently, FIG. 1 illustrates a configuration composed of individual conductive wires coated with a dielectric ceramic which are situated around a fiberglass filter matrix material coated with photocatalytic TiO₂ anatase (or other photocatalytic material). The wires are connected to a high voltage power supply in order to create a type of atmospheric pressure plasma known as a dielectric barrier discharge (DBD). The photocatalytic anatase TiO₂ under (and enveloped on all sides) the plasma generated UV light generates electron-hole pairs on the surface of the photocatalyst for production of OH radicals and other radicals that can oxidize or breakdown microorganisms and/or that can mineralize chemical agents by forming elemental oxides and other high vapor pressure low molecular weight oxidation products. In complement, the atmospheric plasma a) generates UV light for activating the anatase TiO₂ (photonic wavelength less than 380 nm or electron bombardment in excess of 3.2 eV or at an energy above the bandgap of the photocatalyst in general), b) produces energetic and chemically active species which too can break down microorganisms or chemical agents, c) produces germicidal light (less than 260 nm) and d) creates a super-hydrophilic surface across the entire photocatalytic surface area exposed to the plasma thus increasing the surface tension to spread any liquid droplets or aerosols into a thin layer, thus enhancing the ability to decompose the pathogens. Many biological pathogens, like bacteria and viruses and specifically SARS-CoV-2 are known to spread from close person-to-person contact through respiratory droplets or aerosols, according to the Centers for Disease Control and Prevention (CDC). Accordingly, the present invention's creation of a super-hydrophilic surface on the catalyst can effectively take those semi-spherical aerosolized droplets and spread them out into a very thin layer across the photocatalytically activated surface so they can be rapidly mineralized by all aspects of the synergistic actions of the plasma filter. The effect of creating a filter with a super hydrophilic surface acts to drastically increase the probability that when a particle, droplet, or microbial entity impinges on the surface that the enhanced surface energy will capture and spread out the droplet. This is in contrast to a hydrophobic or super hydrophobic surface which would tend to shed the particle or droplet, relatively intact, and avoid deactivation.

In one embodiment of the invention, TiO₂ anatase, as a photocatalytic agent (or other photocatalytic material), produces reactive chemical species which are capable of deactivating biological and chemical agents. The regenerative characteristics of the anatase photocatalytic coating on the fiberglass filter material will prevent the re-release of contaminating agents and maximize the time between filter changes with the possibility of eliminating the need for filter changes entirely. In general, all organic molecules can be oxidized or mineralized to simple oxides and nitrides. The water vapor in the supplied air to the filter will constantly replenish the water vapor absorbed onto the catalytic surface. The catalytic surface in combination with the UV light will dissociate the water on the surface forming OH groups. Pathogenic particles, liquid aerosols, or gaseous species that impinge on the surface covered in activated hydroxyls and other plasma radicals and metastables will be effectively oxidized into simple oxide compounds like H₂O or CO₂. This effectively converts all of the organic chemical or biological pathogens into non-toxic gases which leave the surface with minimal, if any, residue. Additionally, the atmospheric plasma produces reactive species and charged particles for disinfecting and additionally generates some germicidal UV light at and below a wavelength of 260 nm. In one embodiment of the invention, these aspects (plasma activated decontamination, photocatalysis, and UV radiation) along with the use of filter media to physically stop the pathogens/chemical agents are all individually and collectively effective in mineralizing chemical and biological agents in a long life air filtration device and effective at breaking down bacterial and viral agents in order to sterilize the gas flow.

In one embodiment, this inventive approach avoids necessarily photo-activating the anatase in the filtration medium (e.g., in filter mat 16) with conventional UV sources (where only line of sight activation is possible and the amount of available surface area to activate is a fraction of the physical surface area). Instead, the atmospheric pressure plasma discharges used in the present invention provide a stable, intense, glow discharge, thereby generating UV light internally to the plasma filter.

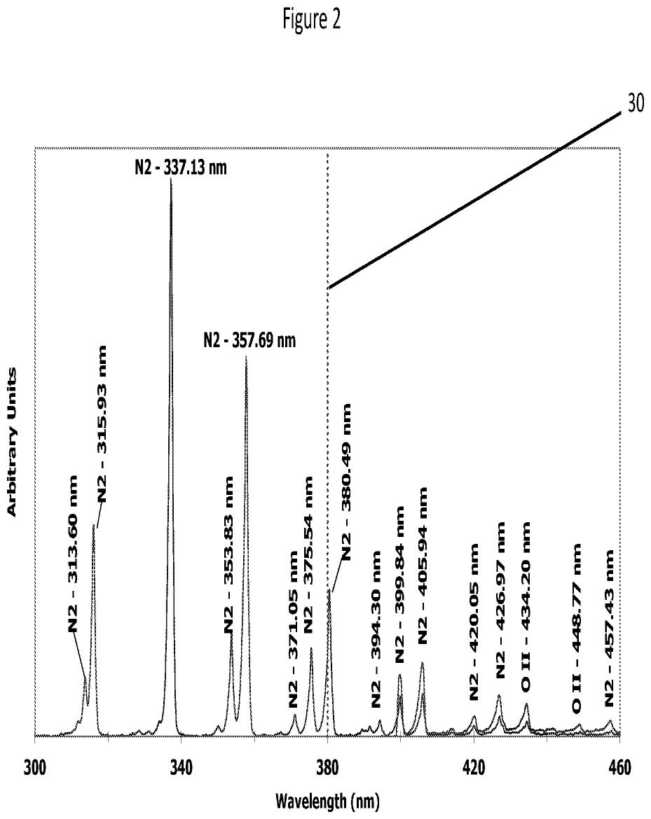

In one embodiment, this inventive approach takes advantage of the combination of atmospheric pressure plasma and the photocatalytic properties of anatase to produce an environment of extreme oxidation for air purification. The efficiency of plasma filter 2 is increased by the activation of the anatase coating by the atmospheric plasma. UV light that otherwise may not have contributed to the decontamination is now an integral part of the system, being responsible for maintaining an activated photocatalyst. Spectra from plasma filter 2 obtained at atmospheric pressure in air is shown in FIG. 2 which shows emission below 380 nm, the cut-off wavelength for anatase activation.

This spectrum in FIG. 2 illustrates the capability of plasma filter 2 operated with an air plasma to generate UV light with a high enough energy to photocatalytically activate the anatase photocatalyst. Anatase requires UV light irradiation below a wavelength of 380 nm to attain a full photocatalytic effect. In FIG. 2, a marker line 30 at 380 nm shows that much of the light given off by the plasma is below the required wavelength for anatase activation and thus sufficient to photoactivate the anatase (or other photocatalytic material). A substantial portion of the UV generated in an air atmospheric plasma is available for anatase activation. The use of photocatalytically activated TiO₂ anatase provides a regenerative, continuous, and stable mechanism for mineralization of microorganisms and chemical agents.

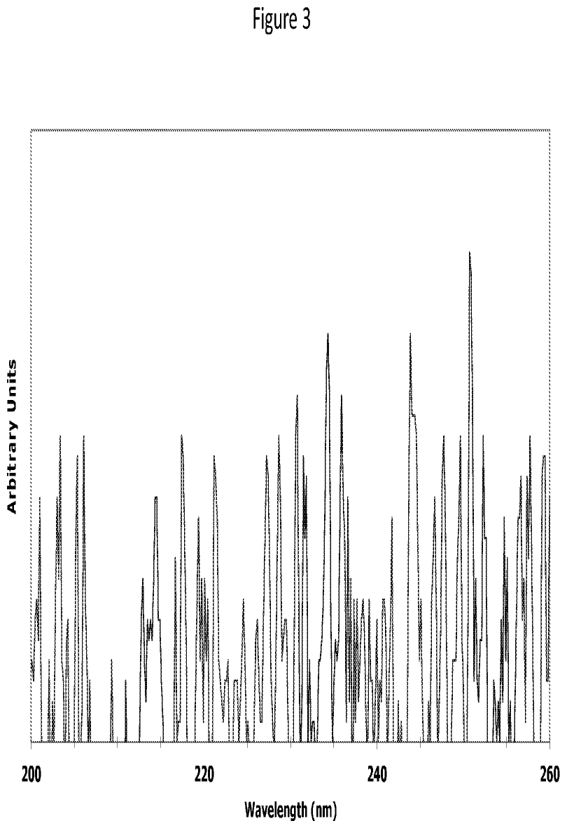

Meanwhile, the plasma optical emission spectra shown in FIG. 3 illustrates some of the germicidal UV light generated by plasma filter 2 operated with an air plasma. The germicidal UV light (i.e., the spectrum shown in FIG. 3) will contribute to the deactivation and sterilization of biological agents including viral and bacterial agents. Plasma filter 2 operated with an air plasma also produces a number of different nitrogen and oxygen activated species. These nitrogen and oxygen excited species are highly chemically reactive and will act to further mineralize organics, such as gaseous nerve agents and other biological agents that enter into the filter media and the active plasma volume.

In one embodiment of the invention, plasma filter 2 can capture 99.999+% of particles due to the near-unity sticking coefficient provided by the plasma enveloped, photocatalytically activated surface of the filtration medium while exhibiting less airflow resistance as compared to a conventional high efficiency particulate air (HEPA) filter. In one embodiment of the invention, plasma filter 2 is self-cleaning and regenerable, maximizing the time required for necessary filter changes. In one embodiment of the invention, plasma filter 2 can neutralize chemical and biological contaminants thereby preventing these agents from being re-released into the atmosphere even during filter change procedures. In one embodiment of the invention, plasma filter 2 represents a scalable design which can address key protection requirements. The filter is scalable in that it is possible for example to create a single plasma filtration panel, 1 ft×1 ft for example. This panel could work as a stand-alone filter or an array of these filter panels could be ganged together to create a larger area filter. In one embodiment of the invention, with plasma filter 2, chemical and biological airborne contaminants can be deactivated and mineralized, thus achieving improved standards in air filtration technology.

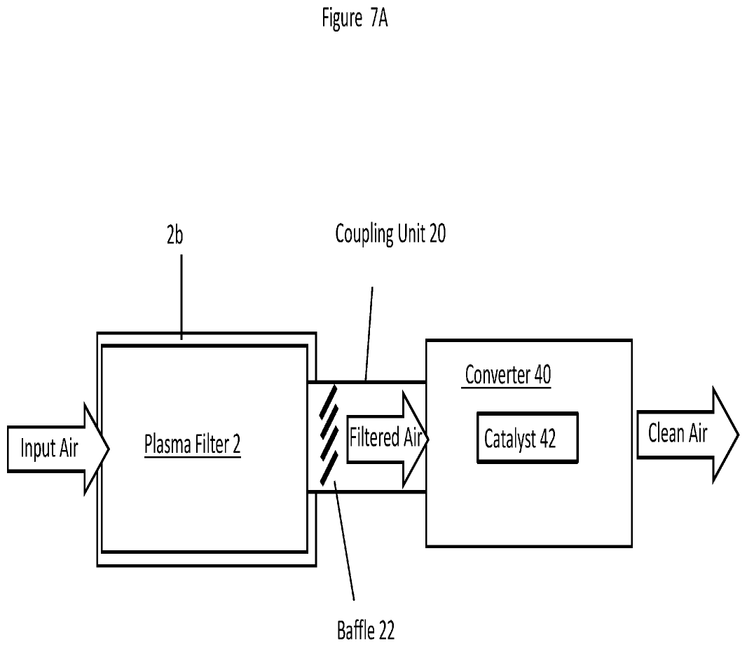

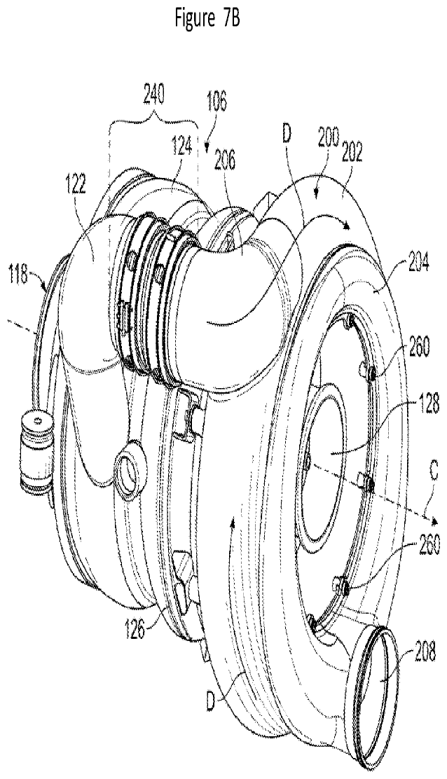

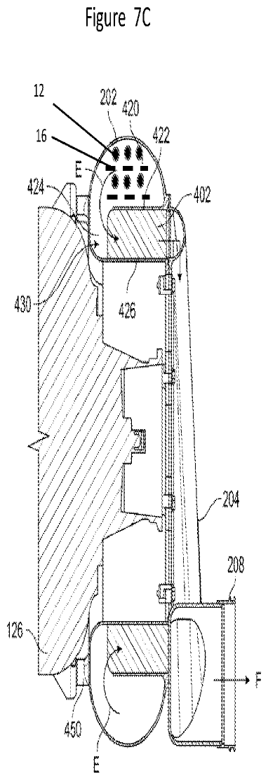

In one embodiment of the invention, as shown in FIG. 7, accompanying plasma filter 2 is a converter 40 used for example to reduce/remove an amount of ozone which is likely generated by the plasma UV light in plasma filter 2 and therefore likely present in the exiting filtered air. In this embodiment, the plasma filter 2 shown in FIG. 1 would be disposed in an enclosure 2b. As shown in FIG. 7, input air supplied to plasma filter 2 flows inside a coupling unit 20 and is supplied into converter 40 containing catalyst 42 (which in this example converts ozone into oxygen), with the “clean air” exiting the system shown in FIG. 7A having been plasma filtered to remove the aforementioned chemical and biological airborne contaminants and having been cleaned of ozone. Catalyst 42 can include metals or metal oxides of palladium (Pd), nickel (Ni), platinum (Pt), rhodium (Rh), gold (Au), iridium Or), silver (Ag), manganese (Mn), cobalt (Co), iron (Fe), and copper (Cu).

Coupling unit 20 is preferably a sealed duct carrying the effluent from the enclosure 2b of plasma filter 2 into ozone converter 40 (also a sealed unit). Coupling unit 40 may contain a baffle 22 including TiO₂ or other photocatalyst which can be used to further increase the surface area of UV exposed photocatalytic material. Baffle 22 may contain other media to block/absorb UV light and/or prevent UV light from interacting with any materials (such as the catalyst 42) in ozone converter 40.

One technique to remove ozone is to catalytically decompose the ozone molecules to form oxygen molecules according to the reaction represented by the formula:

2O₃→3O₂

Catalytic decomposition of ozone is known in the art. U.S. Pat. No. 9,205,402 (the entire contents of which are incorporated herein by reference) describes an ozone converter for an aircraft. U.S. Pat. No. 10,093,425 (the entire contents of which are incorporated herein by reference) describes an annular ozone converter with a replaceable catalyst core. U.S. Pat. No. 8,394,331 (the entire contents of which are incorporated herein by reference) describes a combined hydrocarbon/ozone converter including a substrate, a metal oxide washcoat and a hydrocarbon converting catalyst, such as platinum. The metal oxide washcoat in the '331 patent comprises an ozone reacting component, such as cobalt oxide, and a non-catalytic component, such as aluminum oxide.

U.S. Pat. No. 10,850,855 (the entire contents of which are incorporated herein by reference) describes an aircraft cabin air system having a first air treatment module and a second air treatment module. The first air treatment module comprises an inlet in fluid communication with an air source, an adsorbent comprising a transition metal oxide or metal organic framework, and an outlet. The second air treatment module comprises an inlet in fluid communication with the first air treatment module outlet, a noble metal catalyst and an outlet that discharges ozone-depleted air.

U.S. Pat. No. 7,604,779 (the entire contents of which are incorporated herein by reference) describes an aircraft environmental control system including a catalytic converter having ozone-destroying capability. The catalyst in the '779 patent included monometallic, or bimetallics in the form of oxide with different valence states or in zero valance metallic state. Such catalyst in the form of oxide(s) included palladium (Pd), nickel (Ni), platinum (Pt), rhodium (Rh), gold (Au), iridium (Ir), silver (Ag), manganese (Mn), cobalt (Co), iron (Fe), and copper (Cu).

These prior art ozone converters and others are suitable for converter 40 shown in FIGS. 7A, 7B, and 7C.

In a preferred embodiment, the amount of ozone in the clean air exiting the ozone converter 40 meets regulatory standards such as for example those mandated by the Federal Aviation Agency (FAA) of less than 100 ppb 3 hour average of O₃ and less than 250 ppb peak O₃. Other regulatory standards apply such as the World Health specifying that the maximum ozone concentration must not exceed 0.24 mg/m³ for 8-h exposure, and the average ozone concentration in 8-h exposure is limited to 0.12 mg/m³. Chinese “indoor air quality standards” (GB/T 18883-2002) also require that the indoor ozone concentration not exceed 0.214 mg/m³.

Presently, there are commercially available filters used in the aircraft industry to remove ozone and volatile organic compounds (VOCs) from air being supplied to passenger cabins. These converters (such as BASF's Deoxo™ aircraft cabin ozone converters and MKS Instruments' OVS ozone gas destruct units are suitable for the present invention.

Plasma Filter Construction

A plasma filter of the present invention utilizes an atmospheric pressure plasma system, electrode array, power supply, and a fiberglass filter mesh weave (or any other inorganic or ceramic filter media or media that is resistant to the effects of the plasma or the photocatalytically activated surfaces).

The following describes one procedure according to the invention for obtaining the filtration mat 16. Commercially available woven and non-woven fiberglass materials (for the filter media) can be coated by a solution deposition method (a sol gel process). To obtain the coating, in a N filled glove box, tetra-isopropyltitanate Ti(OC₃H₇)₄), TPT, DuPont) is diluted into anhydrous ethyl alcohol to obtain a 10 wt % solution. The solution is then stirred at room temperature. Non-woven and woven fiberglass samples are cleaned with acetone and ethyl alcohol in an ultrasonic cleanser and thereafter dipped into the solution for 30 minutes. The samples are slowly be removed from the solution and allowed to dry in an oven for 1 hour at 100° C. The samples are then heat treated in a furnace to promote crystallization. Table I summarizes the TiO₂ deposition conditions.

| | Solution | Furnace | |
|---|---|---|---|
| | Stirring | Temperature | Time in |
| Condition | Time (hr) | (° C.) | Furnace (hr) |
| #1 | 24 | 400 | 1 |
| #2 | 1 | 450 | 1 |
| #3 | 1 | 450 | 2 |

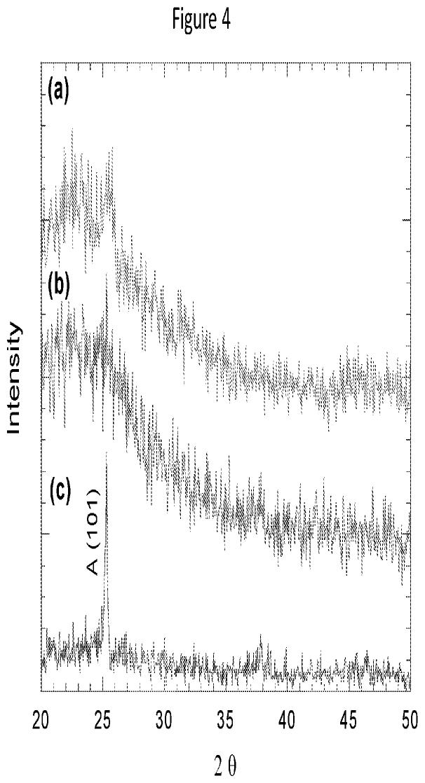

X-ray Diffraction (XRD), (G Flex, Rigaku) with Cu Kα radiation was used to examine the TiO₂ coatings for phase identification. The data collected from the XRD is shown in FIG. 4. Heating for 1 hour at 400° C. and heating for 1 hour at 450° C. were not enough to produce crystalline TiO₂ phase as seen in FIGS. 4 (a) and (b). An anatase phase with (101) orientation was obtained by heating at 450° C. for 2 hours (FIG. 4 (c)).

The following describes one procedure according to the invention for obtaining the dielectric barrier plasma electrode assembly 10 constructed of electrodes surrounded by dielectric materials. Each electrode contains a central conductive core surrounded by approximately 2.5 mm of alumina ceramic. The total thickness of a single electrode is approximately 5 mm. The distance between the dielectrics of each electrode is 2-5 mm. The two (top and bottom) electrode arrays are powered by a power supply capable of supplying 0-30 kVᵣₘₛ and frequencies in the range of 0-50 kHz.

The plasma filter 2 is constructed from three primary components; the dielectric covered electrodes, a fiberglass filter media, and a coating of TiO₂ anatase that is applied onto the fiberglass filter fabric as seen in FIG. 5 showing an example of the dielectric barrier plasma electrode assembly 10 with an interleaved filtration mat 16. In one embodiment of the invention, the TiO₂ fiberglass filter material 16 is woven back and forth between two sets of interdigitated parallel electrode arrays. This arrangement can include multiple number of layers of the filter material 16.

Air Handler utilizing Plasma Filter with Ozone Converter

FIG. 7B is a schematic of air handler of the present invention including an atmospheric plasma filter and ozone converter in an annular configuration. As shown in FIG. 7B, air handler 106 receives an air stream from inlet 118 which in one embodiment of the present invention can include chemical and biological airborne contaminants. Air handler 104 may include a pumping mechanism 106 such as a compressor 124 and/or an optional turbine 126 for driving the compressor. In general, compressor 124 is a mechanical device that raises the pressure of the air received from the inlet 118. Examples of compressor types include centrifugal, diagonal or mixed-flow, axial-flow, reciprocating, ionic liquid piston, rotary screw, rotary vane, scroll, diaphragm, air bubble, etc. Further, compressors can be driven by a motor or the air via the turbine 126. The turbine 126 includes a turbine outlet 128 that may, for example, provide air downstream to air handler 106.

Air that enters the converter inlet 206 fluidly from the compressor outlet 122 is initially provided into an inlet section 202. Air travels in a generally circular path as indicated by arrow D, passes through a core within the converter 200 and enters an outlet housing 204 that includes convener outlet 208. The outlet housing 204 may be removably attached to the inlet housing 202 by one more fasteners 260.

FIG. 7C shows a cross-section taken along axis C of FIG. 7B. Air that enters the inlet housing 202 initially travels in an outer passage 420. The outer passage 420 is at least partially separated from an inner passage 424 by a dividing wall 422 (acting as a baffle). The inner passage 424 is at least partially surrounded by the outer passage and, as such, is disposed radially inwardly from the outer passage. A core 402 is disposed in the inner passage 424. The core removes ozone from air that has left the outer passage 420 and entered the inner passage 424. The movement of air from the outer passage 420 to the inner passage 424 is generally shown by flow arrows E.

As can be seen in FIG. 7B, outer passage 420 contains an atmospheric plasma filter 2 including the dielectric barrier assembly 19 such as shown in FIG. 1 or 5 and illustrated in FIG. 7B, by the presence of dielectric coated electrodes 12 and TiO₂ coated fabric 16, which extend along circumferential paths in outer passage 420.

Plasma Filter Operation

Listeria innocua is a non-spore forming, nonbranching, gram-positive rod that can occur individually or form short chains. Both the pathogentic and innocuous forms of Listeria can survive in many extreme conditions such as high pH, high salt concentrations, and high temperature.

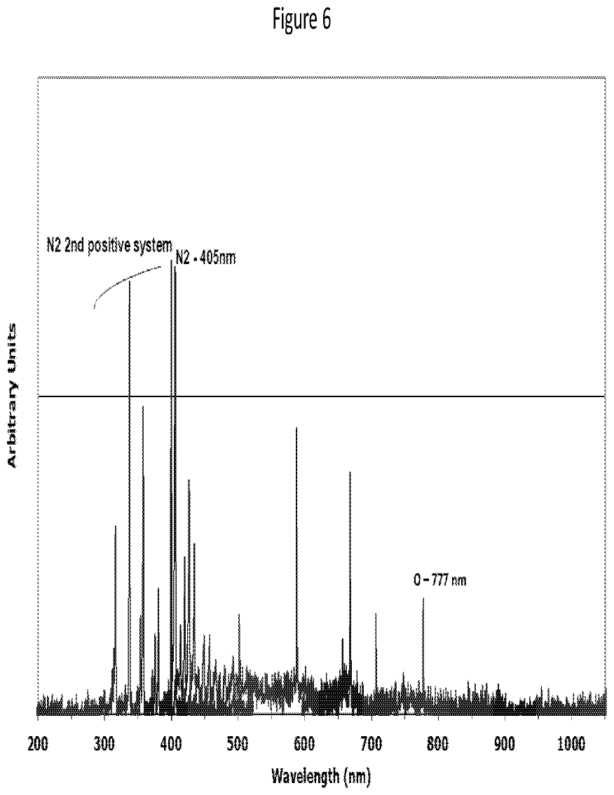

The feasibility experiments for killing a nonpathogenic Listeria innocua by an atmospheric plasma has been demonstrated. An atmospheric plasma used for the experiments was generated by a dielectric barrier discharge. The dielectric barrier discharge setup consisted of two dielectric (Pyrex) plates placed between two electrodes (one driven and one grounded). The voltage input into the plasma was 5500 V and the input current into the plasma was 0.04 A. A TiO₂ anatase filtration mat was not used in in this demonstration. After treatment by the atmospheric plasma, a greater than 5 log reduction was achieved in multiple experiments. A plasma emission spectrum observed in these experiments is provided in FIG. 6 showing N₂ and atomic O emission lines reflective of the gas mixtures used to generate an atmospheric pressure plasma used to reduce the Listeria innocua count. Here, in this example, petri dishes containing nutrient growth agar were inoculated with live bacteria across the surface of the agar. The control samples showed bacterial colonies that covered the petri dish completely in a couple days. The plasma treated petri dishes exhibited a nearly complete destruction of the bacteria in the area where the plasma interacted with the inoculated agar.

Computer Control

It will be understood that the power supply 14 schematically illustrated in FIG. 1 may control more than the power to the electrodes 12. For example, it can be programmed or otherwise configured to control the flow of gas through the plasma filter 2 and to monitor for example the plasma emissions such as those shown in FIGS. 2, 3, and 6 to be sure that UV lines of importance are maintained. In general, power supply 14 comprises a programmed controller.

Accordingly, power supply 14 may include one or more types of user devices, such as user input devices (e.g., keypad, touch screen, mouse, and the like), user output devices (e.g., display screen, printer, visual indicators or alerts, audible indicators or alerts, and the like), a graphical user interface (GUI) controlled by software for display by an output device, and one or more devices can be used for loading media (e.g., logic instructions embodied in software, data, and the like) thereon. Thus, the controller of power supply 14 may include an operating system (e.g., Microsoft Windows® software) for controlling and managing various functions of the power supply 14.

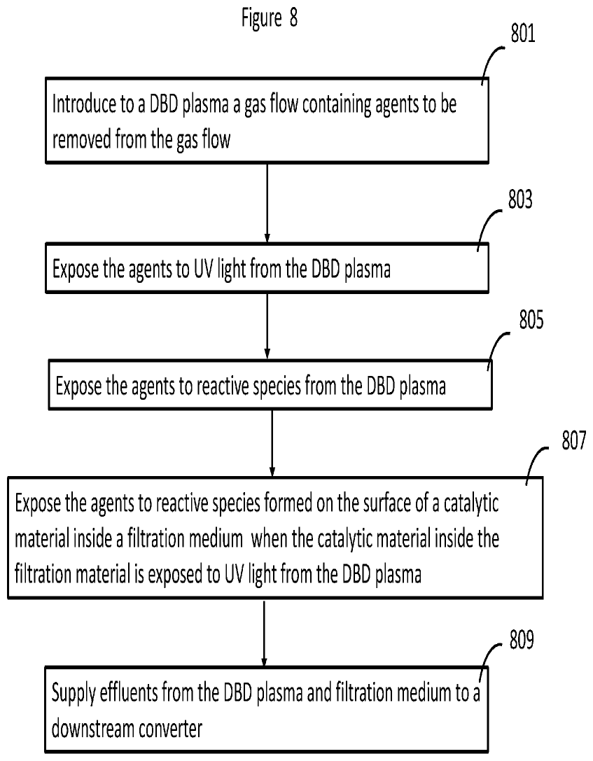

FIG. 8 is a flowchart detailing a method of the present invention for sterilization (and/or disinfection) of a gas flow. At step 701, a gas flow containing agents to be removed from the gas flow is introduced to a dielectric barrier discharge (DBD) plasma (preferably but not necessarily at atmospheric pressure). In this step, a controller such as the controller of power supply 14 can control a pumping rate for supplying a gas from outside the plasma filter 2 to the DBD discharge. In this sense, the DBD may operate a pressure slightly reduced from atmosphere in order to draw gas into the plasma filter 2.

At step 803, the agents in the gas flow are exposed to UV light from the DBD plasma. This exposure can be before or after the gas with the contaminants enters the filtration mat 16. In some embodiments, the contaminants are trapped on the surface of the filtration medium where the trapped contaminants are exposed to the UV light generated by the DBD plasma. In some embodiments, the contaminants are trapped inside voids of the filtration medium where the trapped contaminants are exposed to the UV light generated by the DBD plasma including, but not limited to, UV light generated by plasma inside the voids.

At step 805, the agents in the gas flow are exposed to reactive species from the DBD plasma. In some embodiments, the contaminants are trapped on the surface of the filtration medium where the trapped contaminants are exposed to the reactive species generated by the DBD plasma. In some embodiments, the contaminants are trapped inside voids of the filtration medium where the trapped contaminants are exposed to the reactive species generated by the DBD plasma including, but not limited to, reactive species generated by plasma inside the voids.

Optionally, at step 807, the agents in the gas flow are exposed to reactive species formed on the surfaces of a catalytic material inside a filtration medium when the catalytic material inside the filtration medium is exposed to UV light from the DBD plasma. In some embodiments, the filtration medium is exposed to the UV light generated by the DBD plasma. In some embodiments, the filtration medium is exposed to the UV light generated by the DBD plasma including, but not limited to, UV light generated by plasma inside the voids. The UV light in one aspect of the invention generates reactive OH groups which can interact with the contaminants from the gas flow. Additionally, while the present invention is not limited to the following, the process of the UV light being absorbed by the catalyst (semiconductor materials) generates electron-hole pairs at the surface by absorption of light at or above the energy band gap of the catalyst which in the presence of moisture containing air generates hydroxyl groups on the surface of the catalyst. In one aspect of the invention, the generated hydroxyl groups and other excited species on the surface of the catalyst increases the surface energy of the catalytic material so that for example contaminants contained in aerosol droplets will better wet out to the surface of the photo-activated surfaces of the catalytic material, thereby increasing the sticking coefficient of the aerosols to the photo-activated surfaces of the catalytic material and increasing neutralization of the contaminants. This effect can be enhanced further by the addition of multiple sources of UV light (or any light above the bandgap of the catalyst) from another source such as lamp 15 in FIG. 1 which can be for example a Hg lamp, a Xe lamp, and/or a UV/blue light emitting diode, any of which preferably are contained in UV a transparent housing such as fused silica, quartz, sapphire, or spinel. In one embodiment, other chemical species can be injected into the DBD section or the downstream plasma section in order to create specific chemistries to target a greater range of contaminants to selectively disable a specific chemical or biological agent with a known weakness or susceptibility to a specific chemistry. Biological agents including but not limited to prions, coccidia, spores, mycobacteria, cysts, non-enveloped viruses, trophozoites, gram-negative bacteria, fungi, gram-positive bacteria, and lipid enveloped viruses may have specific resistances to the standard air plasma and photocatalytic disinfection chemistry. In these cases, the injection of another chemical species or compound into the ambient air stream or into specific regions of the filter, such as the DBD plasma region or the downstream region from the plasma, may be beneficial to effectively target and eliminate certain resistant biological or chemical agents.

One example would be the destruction of the bacteria Bacillus pumilus SAFR-032, a spore forming bacteria that is known to have enhanced resistance to peroxides. UV radiation and chemical disinfection in general. It has been shown that Bacillus pumilus SAFR-032 is effectively disinfected with chlorine containing disinfectant species. The addition of a halogen gas, such as chlorine, into the DBD section of the plasma filter would enhance the disinfection of the Bacillus pumilus SAFR-032 by exposing the bacteria to the activated chlorine species formed in the reactive plasma environment. This same principle could also be applied to effectively eliminate specific chemical warfare agents by selectively targeting known chemical weaknesses in the chemical structure of these agents to allow effective neutralization of those chemical agents. Other pollutants or materials that are introduced into the plasma filter can likewise be specifically targeted, if required, with the prudent selection of additional chemistry injected into various regions of the plasma filter system embodied in this invention.

In one embodiment, agents in the gas flow are exposed to high surface energy surfaces produced by the UV catalyzed reaction. These high surface energy surfaces have a high sticking coefficient and wet-out on the catalytic surface maximizing the exposure of the agents to the activated surface area. Surface energy enhancement from exposure to an atmospheric plasma and/or a photocatalytically activated surface will vary depending on the intrinsic surface energy of the material surfaces. In general, an increase of surface energy can be expected when the surface is activated by a plasma process and/or a photocatalytically activated process. With high energy surfaces exhibiting a total surface energy of 60-1000+ dynes/cm.

Optionally, at step 809, the effluents from the DBD plasma and filtration medium (e.g., the plasma filtration medium 2) can be supplied to an ozone converter (e.g., the ozone converter 40 in FIG. 7 and described above) to convert ozone in the effluent from the plasma filtration medium 2 into oxygen.

While the steps described above and depicted in FIG. 8 are depicted in a sequential manner for the purpose of this figure, the present invention is not so limited. For example, the filtration medium may be used to first capture the agents under a no-plasma condition, and afterwards the plasma could be initiated producing the UV light, plasma species, and indirectly to the reactive species from the catalytic material inside the filtration medium.

It will be understood that one or more of the processes, sub-processes, and process steps described herein may be performed by hardware, firmware, software, or a combination of two or more of the foregoing, on one or more electronic or digitally-controlled devices for example adjusting the flow rate of the gas introduced to plasma filter 2, and/or the power dissipated by the plasma filter 2, and/or the mixture of gas supplied to the plasma filter. The software may reside in a software memory (not shown) in a suitable electronic processing component or system such as, for example, the power supply 14 schematically depicted in FIG. 1 and discussed above. The software memory may include an ordered listing of executable instructions for implementing logical functions (that is, “logic” that may be implemented in digital form such as digital circuitry or source code, or in analog form such as an analog source such as an analog electrical, sound, or video signal). The instructions may be executed within a processing module, which includes, for example, one or more microprocessors, general purpose processors, combinations of processors, digital signal processors (DSPs), or application specific integrated circuits (ASICs). The examples of systems described herein may be implemented in a variety of configurations and operate as hardware/software components in a single hardware/software unit, or in separate hardware/software units.

The executable instructions may be implemented as a computer program product having instructions stored therein which, when executed by a processing module of an electronic system (e.g., the controller of power supply 14), direct the electronic system to carry out the instructions. The computer program product may be selectively embodied in any non-transitory computer-readable storage medium for use by or in connection with an instruction execution system, apparatus, or device, such as a electronic computer-based system, processor-containing system, or other system that may selectively fetch the instructions from the instruction execution system, apparatus, or device and execute the instructions. In the context of this disclosure, a computer-readable storage medium is any non-transitory means that may store the program for use by or in connection with the instruction execution system, apparatus, or device. The non-transitory computer-readable storage medium may selectively be, for example, an electronic, magnetic, optical, electromagnetic, infrared, or semiconductor system, apparatus, or device. A non-exhaustive list of more specific examples of non-transitory computer readable media include: an electrical connection having one or more wires (electronic); a portable computer diskette (magnetic); a random access memory (electronic); a read-only memory (electronic), an erasable programmable read only memory such as, for example, flash memory (electronic); a compact disc memory such as, for example, CD-ROM, CD-R, CD-RW (optical): and digital versatile disc memory, i.e., DVD (optical).

It will also be understood that the term “in signal communication” as used herein means that two or more systems, devices, components, modules, or sub-modules are capable of communicating with each other via signals that travel over some type of signal path. The signals may be communication, power, data, or energy signals, which may communicate information, power, or energy from a first system, device, component, module, or sub-module to a second system, device, component, module, or sub-module along a signal path between the first and second system, device, component, module, or sub-module. The signal paths may include physical, electrical, magnetic, electromagnetic, electrochemical, optical, wired, or wireless connections. The signal paths may also include additional systems, devices, components, modules, or sub-modules between the first and second system, device, component, module, or sub-module.

More generally, terms such as “communicate” and “in . . . communication with” (for example, a first component “communicates with” or “is in communication with” a second component) are used herein to indicate a structural, functional, mechanical, electrical, signal, optical, magnetic, electromagnetic, ionic or fluidic relationship between two or more components or elements. As such, the fact that one component is said to communicate with a second component is not intended to exclude the possibility that additional components may be present between, and/or operatively associated or engaged with, the first and second components.

Exemplary Statements of the Invention

The following numbered statements of the invention set forth a number of inventive aspects of the present invention:

Statement 1. A plasma filter for treating a gas flow therethrough, comprising:

a dielectric barrier plasma electrode assembly including a plurality of electrodes having a dielectric barrier layer coated thereon, the dielectric barrier plasma electrode assembly configured to produce an atmospheric pressure plasma; the plurality of electrodes (e.g., forming a supporting grid or other supporting structure);

a filtration medium disposed on or between the electrodes; and

a catalytic material formed on the surface of the filtration mat.

wherein, upon operation of the plasma filter,

the plasma infiltrates voids in the filtration medium, and

the gas flow through the filtration medium t a) is exposed to reactive species of the plasma, b) interacts with the catalytic material on the filtration medium, and c) is exposed to ultraviolet light generated from the plasma. Treatment of the gas flow can include but is not limited to sterilization and/or disinfection of the gas flow where harmful contaminants in the gas flow are removed from the gas.

Statement 2. The filter of statement 1, comprising an air supply configured to provide ambient air to the plasma, wherein at least one component of the air in the plasma generates the ultraviolet light.

Statement 3. The filter of any of the statements above, wherein the filtration medium comprises strands of fiberglass coated with mostly anatase crystalline phase titanium dioxide (TiO₂)(that is an titanium dioxide surface where at least 50% of the titanium dioxide is in the anatase crystalline phase with the remainder constituents being rutile phase or amorphous.) While called out here as a fiberglass medium, this medium can be any material that is resistant to the action of the plasma and the activated photocatalyst. Generally, this medium could be a metal, metal oxide or semiconducting oxide, and in one embodiment could be a membrane comprised of oxidation resistant polymer nanofibers such as Teflon or polyphosphazene.

Statement 4. The filter of any of the statements above, wherein the gas flow is sterilized by flow through the plasma discharge and through the filtration medium.

Statement 5. The filter of any of the statements above, wherein the filtration medium has an opening size to capture aerosols (or other entrained materials in the gas flow) having a diameter between 10-100 nanometers, or between 100-1000 nanometers or between 1-50 microns, or between 50-100 microns, or wherein entrained materials in the gas flow are captured by the action of the entrained materials impinging and sticking to surfaces of the filtration medium or wherein both of these mechanisms (entrapment and sticking) used to capture material entrained in the gas flow. In this aspect of the invention, multiple layers or thicknesses of this filter medium may be stacked to increase or decrease the thickness of the medium, as required for the specific application, to increase the probability of an aerosol or particle coming in contact with the surface of the filtration medium. Accordingly, the filtration medium can capture 99.999+ % of particles due to the near-unity sticking coefficient provided by the plasma enveloped, photocatalytically activated surface of the filtration medium while exhibiting less airflow resistance as compared to a conventional high efficiency particulate air (HEPA) filter. Sticking coefficient describes the ratio of number of particles that adsorb or “stick” to a surface to the total number of particles that impinge on a surface at some time period. Values vary between 0 (zero), where no particles stick, to 1 (one) where all particles stick to the surface. Alternatively, the fiber medium can comprise a non-woven nanofiber material that may have 2-50 nanometer diameter fibers and pores less than 50 nanometers wide, produced for example by electrospinning.

Statement 6. The filter of any of the statements above, wherein the filtration medium is disposed at least between one powered electrode and one grounded electrode. As noted above, alternating electrodes of the plurality of electrodes could be powered with the other electrodes being grounded. Alternatively, individual electrodes of the plurality of electrodes may be driven separately and not necessarily with a ground reference, or individual electrodes of the layers of electrodes may be driven potentially out of phase with one another.

Statement 7. The filter of any of the statements above, wherein the filtration medium comprises multiple sheets of a filter material coated with a photocatalytic material, and the multiple sheets are disposed on or between the electrodes. In one embodiment, a low density foam comprised of an insulating or semiconducting material with an interconnected pore structure that is wash coated with nanophase TiO₂ and sintered or in other way adhered onto the surfaces of the foam can be used as the filter material. In one embodiment, foam being comprised of silica (such as an aerogel) or another low density inorganic foam can be used as the filter material.

Statement 8. The filter of any of the statements above, further comprising a programmed controller configured to at least control power to the electrodes.

Statement 9. The filter of any of the statements above, wherein the controller is programmed to operate the plasma when the gas flow contains agents to be removed from the gas flow.

Statement 10. The filter of statement 9, wherein the controller is programmed to control a pumping rate for supplying the gas flow to the plasma.

Statement 11. The filter of statement 10, wherein the controller is programmed to control a power supplied to the plasma.

Statement 12. The filter of statement 11, wherein the controller is programmed to control the power supplied to the plasma such that the plasma infiltrates voids in the filtration medium

Statement 13. The filter of statement 11, wherein the controller is programmed to monitor plasma emissions for UV emissions.

Statement 14. The filter of any of the statements above, wherein the catalytic material comprises a sol-gel deposited TiO₂ layer on strands of the filtration material, the TiO₂ layer heat treated to comprise an anatase crystalline phase of TiO₂.

Statement 15. The filter of any of the statements above utilized in air handlers.

Statement 16. The filter of statement 15, wherein the air handler is included in at least one of more of an aircraft, a ground vehicle, a maritime vessel, a submarine, a bunker, a housing unit, barracks, classroom, conference room, recreation room, gym, spa, clinic, or other units of confined air space for the occupants thereof.

Statement 17. The filter of statement 16, wherein the air handler is utilized to treat chemical and/or biological agents or other harmful agents in the gas flow such as for example a viral agent and/or a bacterial agent.

Statement 18. A method for sterilization (and/or disinfection) of a gas flow, comprising:

introducing to a dielectric barrier discharge (DBD) plasma a gas flow containing agents (e.g. harmful agents) to be removed from the gas flow;

exposing the agents to UV light from the DBD plasma:

exposing the agents to reactive species from the DBD plasma;

exposing the agents to reactive species from a catalytic material inside a filtration medium when the catalytic material inside the filtration medium is exposed to UV light from the DBD plasma, and optionally

supplying effluents from the filtration medium and the DBD plasma to a downstream converter (such as an ozone converter to generate benign species. In one embodiment, the converter receiving effluent from the filtration medium and the DBD plasma can abate pollutants in the effluent which might occur for example when the filtration medium is itself loaded with contaminants which result in undesirable gases other than just ozone being present in the effluent. In one embodiment, the downstream converted could be a carbon monoxide converter. In yet another embodiment a secondary DBD plasma and secondary filtration medium could be used to provide the further and possibly selective conversion of the undesirable products from the first stage of the filter.

Statement 19. The method of statement 18, wherein exposing the agents to UV light from the DBD plasma comprises exposing the agents to light having a wavelength of 260 nm or shorter.

Statement 20. The method of any of the method statements above, wherein the exposing the agents to reactive species from a catalytic material inside a filtration medium comprises exposing the catalytic material to light having a wavelength of 380 nm or shorter.

Statement 21. The method of any of the method statements above, wherein exposing the catalytic material to light having a wavelength of 380 nm or shorter produces OH radicals for reaction with the agents. In one embodiment, the light needs to have a wavelength only necessary to generate electron-hole pairs across the bandgap of the catalytic materials. For some types of doped TiO₂, such as nitrogen doped TiO₂, the excitation light can be in the violet-blue range. In one embodiment, other photocatalysts having even lower bandgaps can be activated by light in other portions of the visible spectrum, 400 nm-700 nm. In one embodiment, the electrical current flowing through the electrodes and into the semiconducting photocatalytic layer on top of the dielectric layer and then into the plasma may itself generate electron-hole pairs, providing an alternative way to activate the catalytic material, not requiring light or further enhancing the excitation of the catalytic layer.

Statement 22. The method of any of the method statements above, wherein the contaminants in the gas flow comprise at least one of a viral agent, a bacterial agent, and a chemical agent or more generally a biological agent.

Statement 23. The method of any of the method statements above, wherein the DBD plasma infiltrates voids in the filtration medium such that UV light generated from the DBD plasma in the voids activates the catalytic material inside the filtration medium.

Statement 24. A plasma filter and downstream converter system for treating a gas flow therethrough, comprising
a) a plasma filter enclosure containing the component of the plasma filter of statements 1-17, that is:
   a dielectric barrier plasma electrode assembly including a plurality of electrodes having a dielectric barrier layer coated thereon, the dielectric barrier plasma electrode assembly configured to produce an atmospheric pressure plasma;
   a filtration medium disposed on or between the electrodes of the supporting grid; and
   a catalytic material formed on the surface of the filtration medium.
b) a coupling unit connected to the plasma filter enclosure; and
c) a downstream converter connected to the coupling unit and containing for example a catalyst which produces from the effluent a more benign species for example by converting ozone into oxygen,
   wherein, upon operation of the plasma filter and the downstream converter,

the gas flow through the filtration medium a) is exposed to reactive species of the plasma, b) interacts with the catalytic material on the filtration medium, and c) is exposed to ultraviolet light generated from the plasma, and

effluents from the plasma filter are supplied to the downstream converter.

Statement 25. The plasma filter and downstream converter system of statement 24 including any of the filter components recited in any of statements 2-17.

Statement 26. The plasma filter and downstream converter system of statement 24 utilizing any of the method steps recited in any of statements 18-23.

Statement 27. The plasma filter and downstream converter system of statement 24 utilized in air handler.

Statement 28. The plasma filter and downstream converter system of statement 27, wherein the air handler is included in at least one or more of an aircraft, a ground vehicle, a maritime vessel, a submarine, a bunker, a housing unit, barracks, classroom, conference room, recreation room, gym, spa, clinic, or other units of confined air space for the occupants thereof.

Statement 29. The plasma filter and downstream converter system of statement 28, wherein the air handler is utilized to treat chemical and/or biological agents or other harmful agents in the gas flow such as for example

Statement 30. A method for treating a gas flow, comprising:

introducing to a dielectric barrier discharge (DBD) plasma a gas flow containing agents to be removed from the gas flow;

exposing the agents to light from the DBD plasma;

exposing the agents to reactive species from the DBD plasma; and

exposing the agents to reactive species from a catalytic material inside a filtration medium when the catalytic material inside the filtration medium is exposed to light from the DBD plasma.

Numerous modifications and variations of the invention are possible in light of the above teachings. It is therefore to be understood that within the scope of the appended claims, the invention may be practiced otherwise than as specifically described herein.

1. A plasma filter for treating a gas flow therethrough, comprising:
a dielectric barrier plasma electrode assembly including a plurality of electrodes having a dielectric barrier layer coated thereon, the dielectric barrier plasma electrode assembly configured to produce an atmospheric pressure plasma;
a filtration medium disposed on or between the electrodes; and
a catalytic material formed on surfaces of the filtration medium.
wherein, upon operation of the plasma filter,
the plasma infiltrates voids in the filtration medium, and
the gas flow through the filtration medium a) is exposed to reactive species of the plasma, b) interacts with the catalytic material, and c) is exposed to light generated from the plasma.

2. The filter of claim 1, comprising an air supply configured to provide ambient air to the plasma, wherein at least one component of the air in the plasma generates ultraviolet light.

3. The filter of claim 1, wherein the filtration medium comprises strands of fiberglass coated with anatase phase titanium dioxide (TiO₂).

4. The filter of claim 1, wherein the gas flow is sterilized by flow through the plasma discharge and through the filtration medium.

5. The filter of claim 1, wherein entrained materials in the gas flow are captured by the entrained materials impinging and sticking to surfaces of the filtration medium.

6. The filter of claim 1, wherein the filtration medium is disposed at least between one powered electrode and one grounded electrode.

7. The filter of claim 1, wherein the filtration medium comprises multiple sheets of a filter material coated with a photocatalytic material, and the multiple sheets are disposed on or between the electrodes.

8. The filter of claim 1, further comprising a programmed controller configured to at least control power to the electrodes.

9. The filter of claim 1, wherein the controller is programmed to operate the plasma when the gas flow contains agents to be removed from the gas flow.

10. The filter of claim 9, wherein the controller is programmed to control a pumping rate for supplying the gas flow to the plasma.

11. The filter of claim 10, wherein the controller is programmed to control a power supplied to the plasma.

12. The filter of claim 11, wherein the controller is programmed to control the power supplied to the plasma such that the plasma infiltrates voids in the filtration medium.

13. The filter of claim 11, wherein the controller is programmed to monitor plasma emissions for UV or other light emissions.

14. The filter of claim 1, wherein the catalytic material comprises a sol-gel deposited TiO₂ laver on strands of the filtration material, the TiO₂ layer annealed or otherwise converted to comprise an anatase crystalline phase of TiO₂.

15. A method for treating a gas flow, comprising:
introducing to a dielectric barrier discharge (DBD) plasma a gas flow containing agents to be removed from the gas flow;
exposing the agents to UV light from the DBD plasma;
exposing the agents to reactive species from the DBD plasma; and
exposing the agents to reactive species formed on a surface of a catalytic material inside a filtration medium when the catalytic material inside the filtration medium is exposed to light from the DBD plasma.

16. The method of claim 15, wherein exposing the agents to UV light from the DBD plasma comprises exposing the agents to light having a wavelength of 260 nm or shorter.

17. The method of claim 15, wherein the exposing the agents to reactive species from a catalytic material inside a filtration medium comprises exposing the catalytic material to light above a bandgap of the catalytic material.

18. The method of claim 15, wherein the contaminants in the gas flow comprise at least one of a viral agent, a bacterial agent, a biological agent, and a chemical agent.

19. The method of claim 15, wherein the DBD plasma infiltrates voids in the filtration medium such that the light generated from the DBD plasma in the voids activates the catalytic material inside the filtration medium.

20. A plasma filter and downstream converter system for treating a gas flow therethrough, comprising
a) a plasma filter enclosure containing components of the plasma filter of claim 1,
b) a coupling unit connected to the plasma filter enclosure; and
c) a downstream converter connected to the coupling unit,
wherein, upon operation of the plasma filter and the downstream converter,
the gas flow through the filtration medium a) is exposed to reactive species of the plasma, b) interacts with the catalytic material on the filtration medium, and c) is exposed to light generated from the plasma, and
effluents from the plasma filter are supplied to the downstream converter.
